(19) United States
(12) Patent Application Publication
(10) Pub. No.: US 2021/0040257 A1
(43) Pub. Date: Feb. 11, 2021

(54) SURFACE FORMULATIONS COMPRISING PHENOLS

(71) Applicant: Zymergen Inc., Emeryville, CA (US)

(72) Inventors: John MCNAMARA, El Sobrante, CA (US); Nicole G. RICAPITO, Greenwhich, NY (US); Alejandra Alvarez ALBARRAN, Union City, CA (US); Joachim C. RITTER, San Rafael, CA (US); Carol KOCH, San Gabriel, CA (US)

(21) Appl. No.: 16/979,788

(22) PCT Filed: Mar. 15, 2019
(86) PCT No.: PCT/US2019/022559

§ 371 Date: Sep. 10, 2020

Related U.S. Application Data
(60) Provisional application No. 62/644,309, filed on Mar. 16, 2018.
(60) Provisional application No. 62/778,857, filed on Dec. 12, 2018.

(51) Int. Cl. C08G 8/04; C09D 161/12; C09J 161/12; C09J 11/06; C09D 7/63

(52) U.S. Cl. CPC C08G 8/04; C09D 161/12; C09D 7/63; C09J 11/06; C09J 161/12

(57) ABSTRACT

The present disclosure describes surface formulations that form a layer on surfaces and comprise a phenol selected from: (I), (II), (III), (IV) or (V), with detailed descriptions of variables herein.

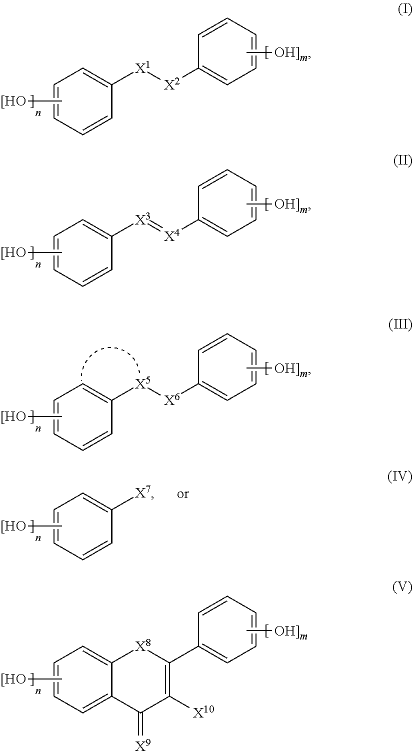

### CROSS-REFERENCE TO RELATED APPLICATIONS

This application claims the benefit of priority to U.S. Provisional Application Nos. 62/644,309, filed Mar. 16, 2018, and 62/778,857, filed Dec. 12, 2018, the contents of which are hereby incorporated by reference in their entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### FIELD OF THE DISCLOSURE

The present invention relates generally to the area of surface modifications with formulations comprising phenols and/or catechols.

### BACKGROUND OF THE DISCLOSURE

Adhesive proteins of marine fouling organisms (e.g., mussels, hydroids, or tubeworms) have attracted considerable interest because of their superior adhesion properties, including rapidity, strength, and versatility, under dry or wet conditions. One of the common structural elements contributing to the adhesive properties of these marine organisms is the incorporation of the phenolic, more precisely, catecholic amino acid 3,4-dihydroxy-L-phenylalanine (DOPA) into the adhesive proteins. Catecholic moieties in DOPA form strong coordination complexes with a host of metal ions, and can form covalent crosslinks in an oxidative environment. These moieties are thus responsible for the excellent wet adhesion properties of marine organisms.

Synthetic polymers incorporating catecholic functionalities for use as surface primers or adhesives are therefore desirable. However, many synthetic challenges exist to access such artificial systems, including the difficulty in preparation of polymers including sensitive catecholic moieties. For example, unprotected catechols can irreversibly crosslink in air at neutral or basic pH, which can limit the shelf life of such materials. Furthermore, existing polymers are made from expensive starting materials. Materials and methods for making surface modifying or adhesive polymers having desirable adhesive properties are therefore needed.

### SUMMARY OF THE INVENTION

In a first aspect, a formulation comprises a phenol. The phenol can be selected from the group consisting of below structures (I), (II), (Ill), (IV), (V) and any combination thereof.

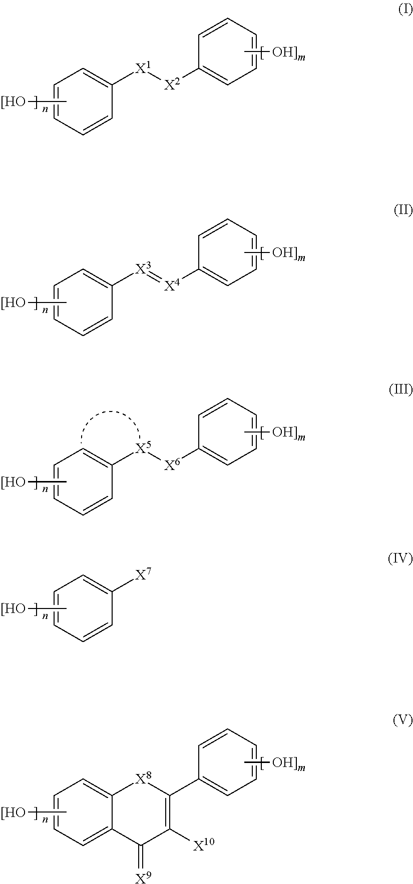

wherein n or m=0, 1, 2, 3, 4, 5, and n+m≥2; X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer; X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer; X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer; X⁴ is ═CR⁴OC(O)(CH₂)ₛ—, ═CR⁴C(O)O(CH₂)ₛ—, ═CR⁴OC(O)(CHR⁴)(CH₂)ₛ—,
═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer; X⁵ is a three, four, or five membered linker forming a fused bicyclic moiety in the phenol; X⁶ is a single bond,
—OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer; X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, a hydroxyl group, or a thiol group; X⁸ and X⁹, for each occasion independently, can be selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl. The phenol can further comprise a property selected from the group of:

(i) the phenol forms a coating when the formulation is applied to a surface and the coating has a water contact angle θ_{C} of equal or greater than 0° and not greater than 65° as determined by ASTM D7334-08,

(ii) the phenol forms an adhesion between a surface and an object having a 90 degree peel resistance as determined according to ASTM D6862-11 of at least 38 N/100 mm,

(iii) the phenol forms a coating when the formulation is applied to a surface and the coating has a tape test rating as determined by ASTM D3359-17 of at least 2B,

(iv) the phenol forms an adhesion between a surface and an object having a lap shear resistance as determined by ASTM D1002, D3163, D3165, or D5868 of at least 0.1 N/mm²,

(v) the phenol forms an adhesion between a surface and an object having a T-peel resistance as determined by ASTM D1876 of at least 60 N/100 mm,

(vi) the phenol forms an adhesion between a surface and an object having a 180 degree peel resistance as determined by ASTM D3330 of at least 38 N/100 mm, and any combination thereof.

In a second aspect, a process for treating a surface comprises:

(a) providing a body;

(b) contacting the surface with a formulation to form a layer, the formulation comprising a phenol selected from the group consisting of

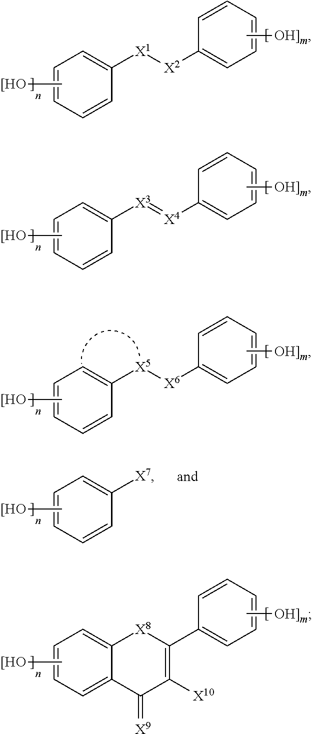

wherein n or m=0, 1, 2, 3, 4, 5, and n+m≥2; X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer; X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer; X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer; X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer; X⁵ is a three, four, or five membered linker forming a fused bicyclic moiety in the phenol; X⁶ is a single bond,

—OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer; X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, a hydroxyl group, or a thiol group; X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl.

The process can further include:

(c) curing the layer to form a coating onto the surface.

The coating can comprise a property selected from

(i) the coating has a water contact angle θ_{C} of equal or greater than 0° and not greater than 65° as determined by ASTM D7334-08, or

(ii) the coating has a tape test rating as determined by ASTM D3359-17 of at least 2B.

In a third aspect, a process for treating a surface comprises

(a) providing a first body; and

(b) contacting the surface with a formulation to form a layer, the formulation comprising a phenol selected from the group consisting of

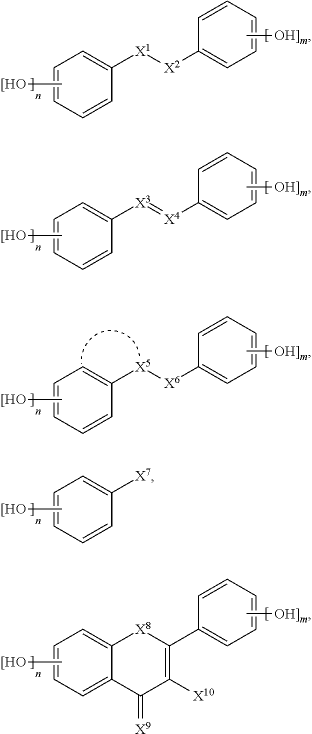

Wherein n or m=0, 1, 2, 3, 4, 5, and n+m≥2; X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer; X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer; X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer; X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer; X⁵ is a three, four, or five membered linker forming a fused bicyclic moiety in the phenol; X⁶ is a single bond, —OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer; X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, a hydroxyl group, or a thiol group; X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl.

The process further comprises step (c) contacting a second body with the layer. The process can further include (d) curing the layer to form an adhesion between the first body and the second body. After curing the layer can have one of the following properties:

(i) the adhesion between the first body and the second body 90 degree peel resistance as determined according to ASTM D6862-11 of at least 38 N/100 mm,

(ii) the adhesion between the first body and the second body has a lap shear resistance as determined by ASTM D1002, D3163, D3165, or D5868 of at least 0.1 N/mm²,

(iii) the adhesion between the first body and the second body has a T-peel resistance as determined by ASTM D1876 of at least 60 N/100 mm,

(iv) the adhesion between the first body and the second body has a 180 degree peel resistance as determined by ASTM D3330 of at least 38 N/100 mm.

### BRIEF DESCRIPTION OF THE DRAWINGS

The present disclosure may be better understood, and its numerous features and advantages made apparent to those skilled in the art by referencing the accompanying drawings.

FIG. 1 illustrates the definition of water contact angle.

FIG. 2 depicts the change of wettability of a surface upon treatment with a catechol.

FIG. 3 depicts the change of wettability of a surface treated with a catechol.

FIG. 4 discloses the change of water contact angle for rosmarinic acid over various dipping time in the absence of an oxidant.

FIG. 5 depicts the lap shear strength of glued metal substrates treated with rosmarinic acid as a primer.

FIG. 6 depicts the water contact angle stability of rosmarinic acid on stainless steel over the course of 30 days.

### DETAILED DESCRIPTION

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. The order in which activities are listed is not necessarily the order in which they are performed.

In this specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Benefits, other advantages, and solutions to problems have been described herein with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

As stated in the Summary of the Invention, all aspects comprise a phenol. The phenol can be selected from the group consisting of below structures (I), (II), (III), (IV), (V) and any combination thereof.

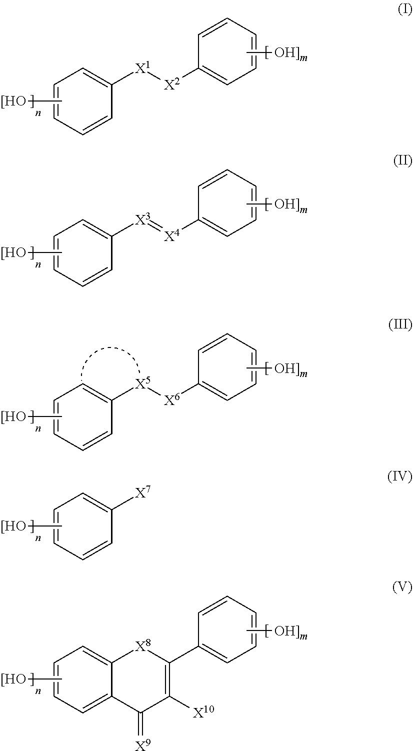

Wherein n or m=0, 1, 2, 3, 4, 5, and n+m≥2; X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer; X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer; X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer; X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer; X⁵ is a three, four, or five membered linker forming a fused bicyclic moiety in the phenol; X⁶ is a single bond, —OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer; X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, a hydroxyl group, or a thiol group; X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl.

In one embodiment, the variables n and m can be selected from one of the following combinations: n=2 and m=0, n=0 and m=2, n=1 and m=1, n=2 and m=1, n=1 and m=2, n=2 and m=2, n=3 and m=0, n=0 and m=3, n=3 and m=1, n=1 and m=3, n=3 and m=2, n=2 and m=3, n=3 and m=3. In another embodiment, the phenol includes one set of two vicinal hydroxyl groups. As understood in the art, the descriptor vicinal defines two functional groups bonded to two adjacent carbon atoms. Two functional groups bonded to two carbon atoms that are not adjacent are referred to as isolated. In yet another embodiment, the phenol includes two sets of two vicinal hydroxyl groups. In one more embodiment, the phenol includes one set of two vicinal hydroxyl groups, and one set of two isolated hydroxyl groups.

In addressing formula I, the phenol can comprise two aromatic groups that are bonded together via an alkylene linker comprising an alkylene moiety X¹ and an ester linker X². X¹ can be CHR¹(CH₂)ₚ—. In one embodiment R¹ can be hydrogen, carboxylate, amino, or hydroxyl and p can be 0, 1, 2, or 3. X² can be —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—. In one embodiment, R² can be selected from hydrogen, a carboxyl group, an amino group, or a hydroxyl group, and q can be 0, 1, 2, or 3.

In addressing formula II, the phenol can comprise two aromatic groups that are bonded together via an alkyne linker comprising two moieties X³ and X⁴ sharing a pi-bond. X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer; X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occurrence selected from hydrogen, a carboxyl group, a sulfonate group, a phosphonate group, an amino group, a hydroxyl group, a thiol group, or a halogen, s is 0 or an integer. In one particular embodiment, a molecule according to formula II is Rosmarinic Acid:

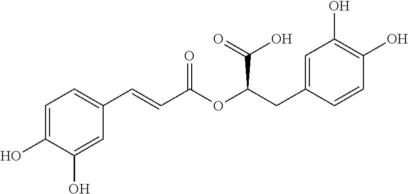 [SMILES: O=C(/C=C/c1ccc(O)c(O)c1)O[C@H](Cc1ccc(O)c(O)c1)C(=O)O]

or a salt thereof. The salt can be an ammonium (NH₄₋ₓRₓ, with R being C₁-C₈ alkyl, and x is 0, 1, 2, 3, or 4), a lithium, a sodium, a potassium, a magnesium, a calcium, or a strontium salt.

In another particular embodiment, a molecule according to formula II is Piceatannol:

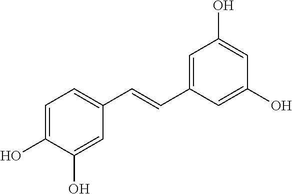 [SMILES: Oc1cc(O)cc(/C=C/c2ccc(O)c(O)c2)c1]

In addressing formula III, one phenol can comprise a bicyclic moiety while the other aromatic group is a single cycle. One example for a compound falling within the scope of formula III is norlaudanosoline:

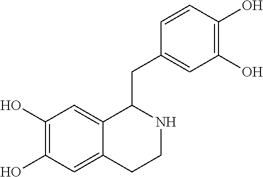 [SMILES: Oc1ccc(CC2NCCc3cc(O)c(O)cc32)cc1O]

In addressing formula IV, the phenol can comprise a single aromatic moiety. In one particular embodiment, a phenol according to formula IV is 3,4-dihydroxybenzaldehyde:

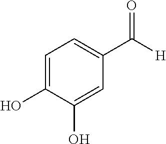

In another embodiment, the phenol according to formula IV can be selected from 4-aminocatechol, 4-nitrocatechol, or 4-chlorocatechol.

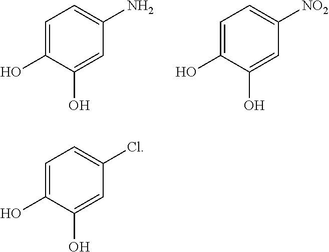

In yet one further embodiment, the molecule according to formula IV is 3,4-dihydroxyphenylglycolic acid or dihydroxymandelic acid:

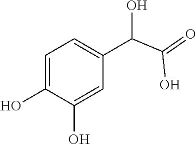 [SMILES: O=C(O)C(O)c1ccc(O)c(O)c1]

Also contemplated are protocatechuic acid or protocatchuic ethyl ester.

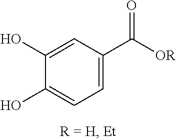

Also within the scope of contemplation are dopamine and dihydroxyphenylacetic acid or ester.

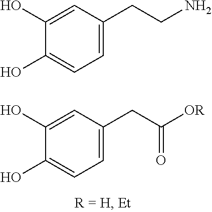

Even another embodiment of the current disclosure are dihydroxyphenyl pyruvates.

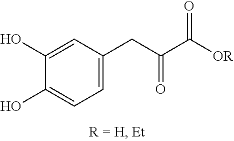

In addressing formula V, the phenol can be a flavone. In one particular embodiment, the phenol can be quercetin

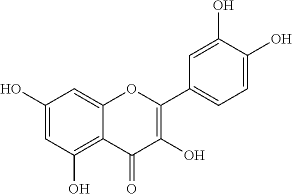 [SMILES: O=c1c(O)c(-c2ccc(O)c(O)c2)oc2cc(O)cc(O)c12]

or luteolin

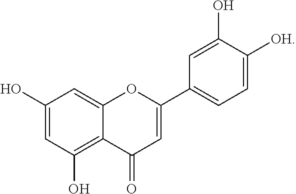 [SMILES: O=c1cc(-c2ccc(O)c(O)c2)oc2cc(O)cc(O)c12]

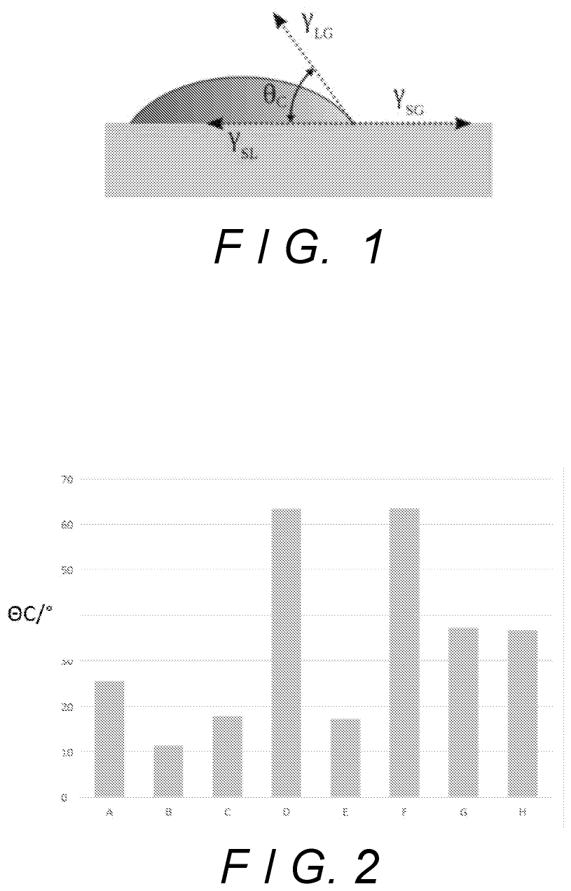

In one embodiment, the phenol forms a coating when the formulation is applied to a surface. The coating can have a water contact angle θ_{C} of equal or greater than 0° and not greater than 65° as determined in accordance with ASTM D7334-08 (2013) “Standard Practice for Surface Wettability of Coatings, Substrates and Pigments by Advancing Contact Angle Measurement.” FIG. 1 depicts a schematic of a liquid drop on a solid surface showing the quantities in the Young-Laplace equation. The theoretical description of contact arises from the consideration of a thermodynamic equilibrium between the three phases: the liquid phase (L), the solid phase (S), and the gas or vapor phase (G) (which could be a mixture of ambient atmosphere and an equilibrium concentration of the liquid vapor). (The “gaseous” phase could be replaced by another immiscible liquid phase.) If the solid-vapor interfacial energy is denoted by γ_{SG}, the solid-liquid interfacial energy by γ_{SL}, and the liquid-vapor interfacial energy (i.e. the surface tension) by γ_{LG}, then the equilibrium contact angle is determined from these quantities by the Young equation:

γ_{SG}−γ_{SL}−γ_{LG} cos θ_{C}=0

In terms of descriptors, water contact angles of 180° are non-wetting, angles between 150° and 180° are termed negligible wetting, angles between 90° and 150° are partial non-wetting, angles between 60° and 90° are partial wetting, angles be greater than 0° and 60° are complete wetting and angles at 0° are spreading. The range of water contact angles greater than 90° are termed hydrophobic, those less than 90° are hydrophilic, wherein angles of 20° and less can be termed superhydrophilic.

In one embodiment, the water contact angle θ_{C} is equal or greater than 0° and not greater than 60°, not greater than 55°, not greater than 55°, not greater than 55°, not greater than 55°, not greater than 55°, not greater than 55°, not greater than 50°, not greater than 48°, not greater than 46°, not greater than 44°, not greater than 42°, not greater than 40°, not greater than 39°, not greater than 38°, not greater than 37°, not greater than 36°, not greater than 35°, not greater than 34°, not greater than 33°, not greater than 32°, not greater than 31°, not greater than 30°, or not greater than 29°.

In yet another embodiment, the water contact angle θ_{C} is greater than 1°, greater than 2°, greater than 3°, greater than 4°, greater than 5°, greater than 6°, greater than 7°, greater than 8°, greater than 9°, greater than 10°, greater than 11°, greater than 12°, greater than 13°, greater than 14°, greater than 15°, greater than 16°, greater than 17°, greater than 18°, greater than 19°, greater than 20°, greater than 21°, greater than 22°, greater than 23°, or greater than 24°.

In one embodiment, the water contact angle θ_{C} is in a range between 1° and 60°, between 2° and 50°, between 5° and 40°, or between 10° and 30°.

In one embodiment, the phenol forms an adhesion between a surface and an object having a 90 degree peel resistance as determined according to ASTM D6862-11 of at least 38 N/100 mm. ASTM D6862-11 entitled “Standard Test Method for 90 Degree Peel Resistance of Adhesives” (11^{th} revision) determines the peel strength of to separate the object from the surface. Unless specified otherwise, the peel strength is determined at room temperature.

In one embodiment, the 90-degree peel resistance is at least 40 N/100 mm, at least 42 N/100 mm, at least 44 N/100 mm, at least 46 N/100 mm, at least 48 N/100 mm, at least 50 N/100 mm, at least 52 N/100 mm, at least 54 N/100 mm, at least 56 N/100 mm, at least 58 N/100 mm, at least 60 N/100 mm, at least 65 N/100 mm, or at least 70 N/100 mm. In one further embodiment, the 90-degree peel resistance can be in a range between 38 N/100 mm and 1 kN/100 mm, such as between 50 N/100 mm and 900 N/100 mm, between 60 N/100 mm and 800 N/100 mm, or 70 N/100 mm and 700 N/100 mm.

In one embodiment, the phenol forms a coating when the formulation is applied to a surface and the coating has a tape test rating as determined by ASTM D3359-17 (“Standard Test Methods for Rating Adhesion by Tape Test”) of at least 2B. In yet another embodiment, the coating has a tape test rating of at least 3B in accordance with ASTM D3359-17, at least 4B in accordance with ASTM D3359-17, or 5B in accordance with ASTM D3359-17.

In one embodiment, the phenol forms an adhesion between a surface and an object having a lap shear resistance as determined by ASTM D1002-10, D3163, D3165, or D5868 of at least 0.1 N/mm². ASTM standards D1002-10 (“Standard Test Method for Apparent Shear Strength of Single-Lap-Joint Adhesively Bonded Metal Specimens by Tension Loading (Metal-to-Metal)”), D3163-01 (2014, “Standard Test Method for Determining Strength of Adhesively Bonded Rigid Plastic Lap-Shear Joints in Shear by Tension Loading”), D3165-07 (2014, “Standard Test Method for Strength Properties of Adhesives in Shear by Tension Loading of Single-Lap-Joint Laminated Assemblies”), and D5868-01 (2014, “Standard Test Method for Lap Shear Adhesion for Fiber Reinforced Plastic (FRP) Bonding”) are test methods for determining bonding characteristics of adhesives joining various materials. The test results are all reported in force per area, e.g. in psi or N/mm².

In one embodiment, the phenol can form an adhesive having a lap shear resistance in accordance with ASTM D1002, D3163, D3165, or D5868 of at least 0.2 N/mm², at least 0.4 N/mm², at least 0.6 N/mm², at least 0.8 N/mm², at least 1 N/mm², at least 1.5 N/mm², at least 2 N/mm², at least 2.5 N/mm², at least 3 N/mm², at least 4 N/mm², at least 5 N/mm², at least 10 N/mm², at least 15 N/mm², at least 20 N/mm², at least 25 N/mm², at least 30 N/mm², at least 35 N/mm², at least 40 N/mm², or at least 45 N/mm².

In one embodiment, the phenol can form an adhesive having a lap shear resistance in accordance with ASTM D1002, D3163, D3165, or D5868 of not greater than 150 N/mm², not greater than 100 N/mm², not greater than 80 N/mm², not greater than 60 N/mm², not greater than 50 N/mm², not greater than 40 N/mm², not greater than 30 N/mm², not greater than 20 N/mm², not greater than 10 N/mm², not greater than 5 N/mm², or not greater than 3 N/mm². In one embodiment, the phenol forms an adhesive having a lap shear resistance in accordance with ASTM D1002, D3163, D3165, or D5868 in the range from 0.1 N/mm² to 80 N/mm², from 0.2 N/mm² to 50 N/mm², or from 0.3 N/mm² to 30 N/mm².

In yet one further embodiment, the phenol forms an adhesion between a surface and an object having a T-peel resistance as determined by ASTM D1876-08 (2015, “Standard Test Method for Peel Resistance of Adhesives (T-Peel Test)) of at least 60 N/100 mm. In yet another embodiment the adhesives formed by the phenols have a T-peel resistance in accordance with ASTM D1876-08 of at least 70 N/100 mm, at least 70 N/100 mm, at least 70 N/100 mm, at least 65 N/100 mm, at least 70 N/100 mm, at least 75 N/100 mm, at least 80 N/100 mm, at least 90 N/100 mm, at least 100 N/100 mm, at least 120 N/100 mm, or at least 150 N/100 mm. In one further embodiment, the adhesives formed by the phenols have a T-peel resistance in accordance with ASTM D1876-08 of not greater than 300 N/100 mm, not greater than 250 N/100 mm, not greater than 200 N/100 mm, not greater than 180 N/100 mm, not greater than 160 N/100 mm, not greater than 140 N/100 mm, not greater than 120 N/100 mm, not greater than 100 N/100 mm, not greater than 95 N/100 mm, not greater than 85 N/100 mm, or not greater than 75 N/100 mm. In yet one further embodiment, the adhesives formed by the phenols have a T-peel resistance in accordance with ASTM D1876-08 in the range of 60 N/100 mm and 300 N/100 mm, in the range of 65 N/100 mm and 200 N/100 mm, in the range of 70 N/100 mm and 150 N/100 mm, or in the range of 75 N/100 mm and 100 N/100 mm.

In yet one further embodiment, the phenol forms an adhesion between a surface and an object having a 180 degree peel resistance as determined by ASTM D3330-04 (2010, “Standard Test Method for Peel Adhesion of Pressure-Sensitive Tape,” Test Method A) of at least 38 N/100 mm, at least 40 N/100 mm, at least 42 N/100 mm, at least 44 N/100 mm, at least 46 N/100 mm, at least 48 N/100 mm, at least 50 N/100 mm, at least 55 N/100 mm, at least 60 N/100 mm, at least 65 N/100 mm, at least 70 N/100 mm, or at least 80 N/100 mm. In one embodiment, the adhesives formed by the phenol have a 180 degree peel resistance in accordance with ASTM D3330-04 of not greater than 250 N/100 mm, not greater than 230 N/100 mm, not greater than 210 N/100 mm, not greater than 190 N/100 mm, not greater than 170 N/100 mm, not greater than 150 N/100 mm, not greater than 130 N/100 mm, or not greater than 110 N/100 mm. In one embodiment, the adhesives formed by the phenol have a 180 degree peel resistance in accordance with ASTM D3330-04 in the range from 38 N/100 mm to 250 N/100 mm, from 45 N/100 mm to 200 N/100 mm, from 50 N/100 mm to 100 N/100 mm.

### Examples

The coating process of catechol-containing compounds involves the oxidation of the catechol moieties to quinones and subsequent polymerization, crosslinking, and deposition onto the substrate surface. This process is normally induced by dissolved O₂ and is facilitated by alkaline conditions and charge screening by salts. Therefore, coating deposition of the molecules was investigated in 600 mM MgCl₂ aqueous solutions buffered at pH 6-9. Each molecule was coated at 1 mg/mL except piceatannol and rosmarinic acid, which were coated at 0.5 mg/mL. Piceatannol was coated at a lower concentration due to its lower solubility in water, and rosmarinic acid was coated at 0.5 mg/mL since this molecule formed thick, inhomogeneous coatings when coated at 1 mg/mL. 3,4-dihydroxybenzaldehyde was coated in a 1:1 molar ratio with ammonium persulfate, a strong oxidant, since this molecule took 72 hours to form a noticeable coating without an oxidizing agent present. The ammonium persulfate made the presence of MgCl₂ unnecessary for coatings to form, so 3,4-dihydroxybenzaldehyde was coated without added MgCl₂ salt. In addition, the other molecules were each screened for their ability to coat without salt. 4-aminocatechol formed coatings at pH 6, 7, and 8 without salt, but the coating at pH 8 was found to be the most uniform and stable.

Under the conditions tested, all 7 molecules were able to form coatings on polycarbonate in 20 hours. 4-aminocatechol was the most versatile in terms of coating formation at a range of pH values, as it was able to form coatings at pH 6-9. It also should be noted that 3,4-dihydroxymandelic acid, 4-nitrocatchecol, 4-chlorocatechol, and 4-aminocatechol when coated without salt each form colorless coatings that become visible only after treatment with a silver nitrate solution. Silver nitrate undergoes a redox reaction with pendant phenols and catechols, leading to elemental silver nanoparticle formation on the coatings, which essentially darkens the coatings and enables coating visualization.

The catechol-containing molecules were then assessed for their ability to coat UHMWPE substrates over 24 hours. Each molecule was coated at its optimal coating pH determined from their coatings on PC. The molecules were all able to coat UHMWPE well. The coatings adhered to the substrates without being able to be rubbed off. 4-nitrocatechol, 4-chlorocatechol, and 4-aminocatechol (coated in the absence of added salt) formed colorless coatings, as they did on the PC substrates.

The molecules were then coated on TiO₂ substrates for 24 hours and characterized by ellipsometry and water contact angle measurements (FIG. 2). The ellipsometry measurements showed that piceatannol, rosmarinic acid, 4-aminocatechol, and 3,4-dihydroxybenzaldehyde coated with ammonium persulfate formed relatively thick coatings around 50 nm. In contrast, 3,4-dihydroxymandelic acid, 4-nitrocatechol, 4-chlorocatechol, and 4-aminocatechol coated without salt formed thinner coatings around 10 nm. It is likely that 3,4-dihydroxymandelic acid, 4-nitrocatechol, and 4-chlorocatechol form thin coatings since these molecules have electron-withdrawing substituents on their catechol moieties. Electron-withdrawing groups slow oxidation of the catechols, resulting in slower coating kinetics. As depicted in FIG. 2, the designations are: Piceatannol (A), Rosmarinic Acid (B), 4-aminocatechol (C), dihydroxybenzaldehyde (+ammonium persulfate) (D), dihydroxymandelic acid (E), 4-nitrocatechol (F), 4-chlorocatechol (G), 4-aminocatechol without salt (H). The contact angles measurements are shown in Table 1:

TABLE 1

| | Water Contact | Standard |
|---|---|---|
| Molecule | Angle | deviation |
| Piceatannol (A) | 25.5 | 0.5 |
| Rosmarinic Acid (B) | 11.4 | 2.0 |
| 4-aminocatechol (C) | 17.8 | 1.0 |
| Dihydroxybenzaldehyde (D) | 63.3 | 0.5 |
| Dihydroxymandelic acid (E) | 17.2 | 1.5 |
| 4-nitrocatechol (F) | 63.4 | 2.0 |
| 4-chlorocatechol (G) | 37.1 | 0.5 |
| 4-aminocatechol w/out salt (H) | 36.7 | 2.0 |

The water contact angle measurements of the coatings show that each of the molecules form hydrophilic coatings, with rosmarinic acid and 3,4-dihydroxymandelic acid forming the most hydrophilic coatings. These coatings had water contact angles of 11.4° and 17.2°, respectively. All of the coatings increased the water contact angle of the super-hydrophilic air plasma-cleaned TiO₂ which has a contact angle of ˜0°.

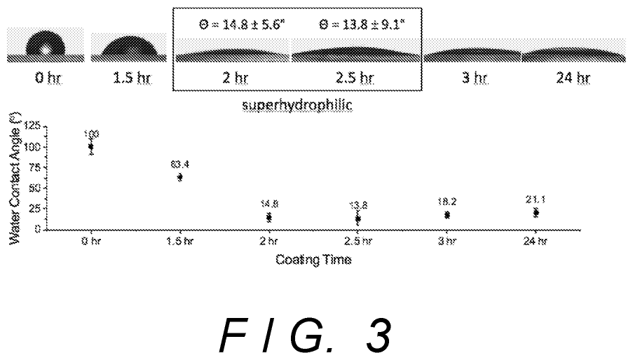

FIG. 3 shows the effect of Rosmarinic Acid catechol coating converting a polydimethylsiloxane surface from a hydrophobic to a superhydrophilic surface in less and maintaining the wettability over time.

Testing on Metal and Polyamide Surfaces

Dip-Coating Procedure

Bis-Tris buffer (0.1M) was prepared with DI water. Magnesium chloride (0.6M) was added to buffer solution. Catechol (from 0.5 to 1.0 mg/ml) was added and stirred to dissolve. When indicated, ammonium persulfate (0.07M) as an oxidant was added. Stainless steel panels that have been cleaned with ethyl acetate and rinsed with DI water are placed in solution for specified period of time. The panels are then removed, rinsed with DI water and allowed to air dry.

The water contact angle is measured using a BTG Surface Analyst.

Cross Hatch Adhesion Test:

A sharp blade was used to score the coating in parallel lines, at 90 degree angles. A pressure sensitive tape is applied using a 21b roller and allowed to dwell for 24 hours. The tape is then peeled away and both the tape and the coated panel are inspected to determine whether the coating has been removed from the panel.

Table 2 shows various catechols screened after dipped for 24 hours on stainless steel.

TABLE 2

| Catechol | BTG Water Contact Angle/deg |
|---|---|
| Piceatannol | 13 |
| Quercetin | 18 |
| Rosmarinic Acid | 19 |
| 3,4-Dihydroxyphenylpyruvate | 19 |
| Dopamine | 25 |
| Luteolin | 27 |
| Dihyroxyphenylacetic acid | 35 |
| 3,4-Dihydroxymandelic acid | 48 |
| Protocatechuic acid ethyl ester | 56 |
| Protocatechuic acid | 63 |
| Chlorocatechol | 67 |
| 3.4-Dihydroxybenzaldehyde | 72 |

Table 3 discloses the water contact angle on various substrates, namely stainless steel (SS), electroless Nickel (Ni), anodized aluminum (Al), and a high performance polyamide (Kalix 9580, obtained from Solvay).

TABLE 3

| Catechol | SS | Ni | Al | Kalix 9580 |
|---|---|---|---|---|
| Uncoated | 78 ± 2 | 86 | 61 | 67 |
| Rosmarinic Acid | 18 ± 5 | 33 | 17 | 16 |
| Dihydroxyphenylpyruvate | 24 ± 10 | 19 | 16 | 17 |
| Dihydroxyphenylacetic | 22 ± 1 | 53 | 28 | 46 |
| acid | | | | |

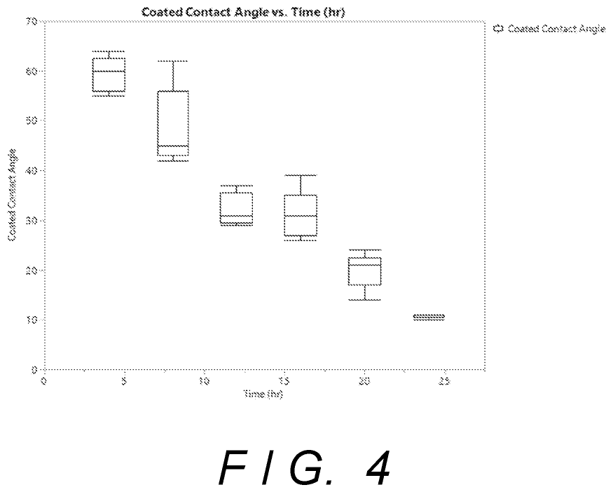

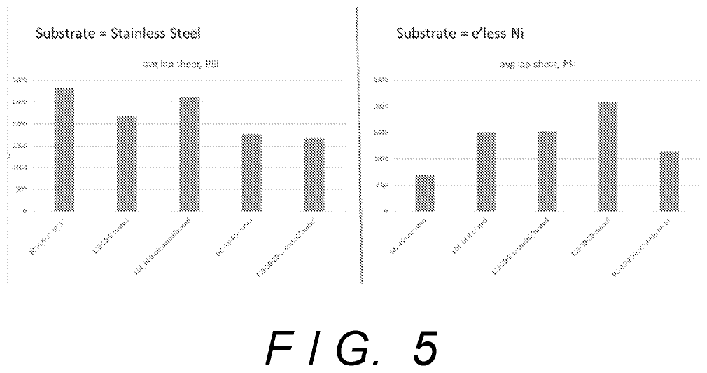

Table 4 shows optimization of the dipping process with Rosmarinic acid (RA) in the presence and absence of ammonium persulfate (APS) as an oxidizer and duration of dipping time. The Table shows water contact angle on stainless steel. FIG. 4 discloses further how the water contact angle changes over 24 hour dipping time in the absence of any oxidant.

TABLE 4

| Condition | 15 min | 30 min | 60 min | 120 min |
|---|---|---|---|---|
| RA (0.05M) | — | — | 60 | 52 |
| no APS | | | | |
| RA (0.05M) | 47 | 27 | 17 | 22 |
| APS (0.07M) | | | | |
| RA (0.025M) | 53 | 27 | 26 | 38 |
| APS (0.07M) | | | | |
| RA (0.05M) | 28 | 42 | 20 | 31 |
| APS (0.035M) | | | | |

Table 5 shows the water contact angle for less water-soluble catechols

TABLE 5

| Catechol | Conditions | WCA |
|---|---|---|
| Luteolin | Standard, 24 hr | 27 |
| Luteolin | 10% acetone | 28 |
| Luteolin | 50% less salt | 16 |
| Quercetin | Standard, 24 hr | 18 |
| Quercetin | 10% acetone | 11 |
| Quercetin | 50% less salt | 17 |
| Quercetin | 50% concentration | 47 |

Table 6 shows further water contact angle of various catechols with and without oxidant on stainless steel.

TABLE 6

| Catechol | Time | | SS | Without Ox., 24 hr |
|---|---|---|---|---|
| Dihydroxybenzoic acid | 120 | min | 36 | 63 |
| Dihydroxymandelic acid | 120 | min | 29 | 48 |
| Dihydroxybenzene | 60 | min | 23 | — |

Adhesion Testing I

Sample Preparation:

Rosmarinic acid was coated for 20 hr on mirror stainless steel and electroless Ni on stainless steel. As adhesive, a conventional two component (2K) polyurethane was used, hand mixed, cured at room temperature. The following specimens were used: uncoated panels, coated panels, mixed (one coated panel and one uncoated panel).

Results

Adhesive strength values were comparable for coated and uncoated panels, but for mixed samples (coated panel/uncoated panel) on SS the adhesive stayed with the uncoated panel, but the primer was not removed from the panel indicating excellent anchorage to stainless steel. This is consistent with the cross hatch adhesion results which show no removal of the coating and therefore excellent anchorage to the panel. For mixed samples (coated panel/uncoated panel) on electroless Ni, the adhesive stayed with the coated panel, indicating stronger adhesion to the primer than the Ni. Similar results were found using an epoxy two part adhesive as well as a methacrylate two part adhesive.

Adhesion Testing II

Sample Preparation

Rosmarinic acid was coated for either 8 hours and 20 hours on stainless steel and electroless Nickel. The water contact angle was measured. Table 7 displays the results.

TABLE 7

| Sample | Sample No. | WCA |
|---|---|---|
| SS, 8 hr | 101-18-8 | 45.6 |
| SS, 20 hr | 101-18-20 | 36.1 |
| Ni, 8 hr | 101-19-8 | 61.1 |
| Ni, 20 hr | 101-19-20 | 32.8 |

A conventional two component epoxy adhesive (3M DP420) was used and cured at room temperature. FIG. 5 summarizes the average lap shear strength in PSI.

Water Contact Angle Stability

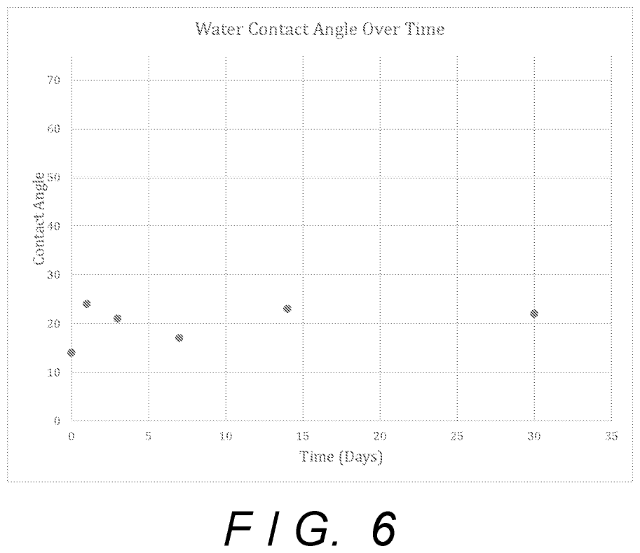

FIG. 6 discloses the water contact angle stability of rosmarinic acid that was dipped for 24 hours on stainless steel.

What is claimed is:

1. A formulation comprising:
a phenol selected from the group consisting of: 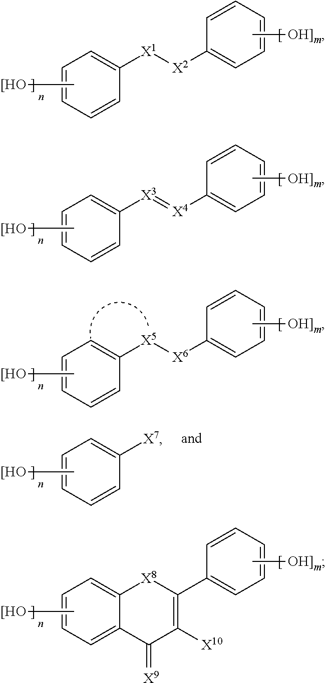
wherein n or m=0, 1, 2, 3, 4, 5, and n+m≥2;
X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer;
X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer;
X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer;
X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer;
X⁵ is a three, four, or five membered linker forming a fused bicyclic moiety in the phenol;
X⁶ is a single bond, a methylene, an ethylene, a propylene, —O(CH₂)ₜ—, —CH₂O(CH₂)ₜ—, —OCH₂(CHR⁵)(CH₂)ₜ—, —CH₂O(CHR⁵)(CH₂)ₜ—, —OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer;
X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, an amino alkylene group, a hydroxyl group, a hydroxyl alkylene group, an alkoxy group, an alkoxy alkylene group, a thiol group, a thiol alkylene group, an alkylthio group, or an alkylthio alkylene group;
X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl;
wherein the phenol further comprises a property selected from the group of
(i) the phenol forms a coating when the formulation is applied to a surface and the coating has a water contact angle θ_{C} of equal or greater than 0° and not greater than 65° as determined by ASTM D7334-08,
(ii) the phenol forms an adhesion between a surface and an object having a 90 degree peel resistance as determined according to ASTM D6862-11 of at least 38 N/100 mm,
(iii) the phenol forms a coating when the formulation is applied to a surface and the coating has a tape test rating as determined by ASTM D3359-17 of at least 2B.
(iv) the phenol forms an adhesion between a surface and an object having a lap shear resistance as determined by ASTM D1002, D3163, D3165, or D5868 of at least 0.1 N/mm²,
(v) the phenol forms an adhesion between a surface and an object having a T-peel resistance as determined by ASTM D1876 of at least 60 N/100 mm,
(vi) the phenol forms an adhesion between a surface and an object having a 180 degree peel resistance as determined by ASTM D3330 of at least 38 N/100 mm.

2. The formulation according to claim 1, further comprising an oxidizing agent.

3. The formulation according to claim 2, wherein the oxidizing agent is selected from the group consisting of oxygen, a peroxide, a peroxydisulfuric acid or salt, a peroxymonosulfuric acid or salt, chlorine, a chlorite, a chlorate, a perchlorate, bromine, a bromite, a bromate, a perbromate, iodine, an iodite, an iodate, a periodate, a permagnate, a perborate, and a chromate.

4. The formulation according to any one of the preceding claims further comprising a salt selected from an alkaline metal halide, earth alkaline halide, or any combination thereof.

5. The formulation according to claim 4, wherein the salt is selected from the group consisting of LiF, LiCl, LiBr, LiI, NaF, NaCl, NaBr, NaI, KF, KCl, KBr, KI, MgF₂, MgCl₂, MgBr₂, MgI₂, CaF₂, CaCl₂, CaBr₂, CaI₂, BaF₂, BaCl₂, BaBr₂, BaI₂, and any combination thereof.

6. The formulation according to any one of the preceding claims, further comprising a diluent.

7. The formulation according to claim 6, wherein the diluent is selected from the group consisting of water, an alkane, an aromatic, an alcohol, an ether, a ketone, an ester, and any combination thereof.

8. The formulation according to any one of the preceding claims, wherein X⁵ is selected from the group consisting of CH—CH₂—CH₂—CH₂—, N—CH₂—CH₂—CH₂—, CH—NH—CH₂—CH₂—, CH—CH₂—NH—CH₂—, CH—CH₂—CH₂—NH—, C═CH₂—CH₂—CH₂—, CH—CH═CH—CH₂, CH—CH₂—CH═CH—, C═CH—CH═CH—, N—CH₂—CH═CH—, N—CH═CH—CH₂—, C═N—CH₂—CH₂—, CH—N═CH₂—CH₂—, CH—NH—CH═CH—, C═CH—NH—CH₂—, CH—CH═N—CH₂—, CH—CH₂—N═CH—, CH—CH═CH—NH—, CH—CH₂—CH═N—, C═N—CH═CH—, C═CH—N═CH₂—, CH═CH—CH═N—, CH—O—CH₂—CH₂—, CH—CH₂—O—CH₂—, CH—CH₂—CH₂—O—, CH—O—CH═CH—, C═CH—O—CH₂—, CH—CH═CH—O—, CH—S—CH₂—CH₂—, CH—CH₂—S—CH₂—, CH—CH₂—CH₂—S—, CH—S—CH═CH—, C═CH—S—CH₂—, CH—CH═CH—S—, N—CH₂—CH₂—, CH—NH—CH₂—, CH—CH₂—NH—, C═N—CH₂—, CH—N═CH—, CH—CH═N—, C═CH—NH—, CH—O—CH₂—, CH—CH₂—O—, C═CH—O—, CH—S—CH₂—, CH—CH₂—S—, C═CH—S—.

9. The formulation according to any one of the preceding claims, wherein the phenol is selected from the group consisting of piceatannol, rosmarinic acid, rosmarinic salt, a norlaudanosoline, 4-aminocatechol, dihydroxybenzaldehyde, dihydroxymandelic acid, 4-nitrocatechol, 4-chlorocatechol, 3,4-dihydroxyphenylpyruvate, epinephrine, norepinephrine, and any combination thereof.

10. The formulation according to any one of the preceding claims, wherein the water contact angle θ_{C} is greater than 1°, greater than 2°, greater than 3°, greater than 4°, greater than 5°, greater than 6°, greater than 7°, greater than 8°, greater than 9°, greater than 10°, greater than 11°, greater than 12°, greater than 13°, greater than 14°, greater than 15°, greater than 16°, greater than 17°, greater than 18°, greater than 19°, greater than 20°, greater than 21°, greater than 22°, greater than 23°, or greater than 24°.

11. The formulation according to any one of the preceding claims, wherein the water contact angle θ_{C} is not greater than 60°, not greater than 55°, not greater than 50°, not greater than 48°, not greater than 46°, not greater than 44°, not greater than 42°, not greater than 40°, not greater than 39°, not greater than 38°, not greater than 37°, not greater than 36°, not greater than 35°, not greater than 34°, not greater than 33°, not greater than 32°, not greater than 31°, not greater than 30°, or not greater than 29°.

12. The formulation according to any one of the preceding claims, wherein the 90-degree peel resistance is at least 40 N/100 mm, at least 42 N/100 mm, at least 44 N/100 mm, at least 46 N/100 mm, at least 48 N/100 mm, at least 50 N/100 mm, at least 52 N/100 mm, at least 54 N/100 mm, at least 56 N/100 mm, at least 58 N/100 mm, at least 60 N/100 mm, at least 65 N/100 mm, or at least 70 N/100 mm.

13. The formulation according to any one of the preceding claims, wherein the lap shear resistance of at least 0.2 N/mm², at least 0.3 N/mm², at least 0.4 N/mm², at least 0.5 N/mm², at least 0.6 N/mm², at least 0.7 N/mm², at least 0.8 N/mm², at least 0.9 N/mm², at least 1.0 N/mm², at least 1.2 N/mm², at least 1.4 N/mm², at least 1.6 N/mm², at least 1.8 N/mm², at least 2 N/mm², at least 2.5 N/mm², at least 3.0 N/mm², or at least 3.5 N/mm².

14. The formulation according to any one of the preceding claims, wherein the T-peel resistance is at least 65 N/100 mm, at least 70 N/100 mm, at least 75 N/100 mm, at least 80 N/100 mm, at least 85 N/100 mm, at least 90 N/100 mm, at least 100 N/100 mm, at least 105 N/100 mm, at least 110 N/100 mm, at least 120 N/100 mm, at least 130 N/100 mm, at least 140 N/100 mm, or at least 150 N/100 mm.

15. The formulation according to any one of the preceding claims, wherein the 180-degree peel resistance is at least 40 N/100 mm, at least 42 N/100 mm, at least 44 N/100 mm, at least 46 N/100 mm, at least 48 N/100 mm, at least 50 N/100 mm, at least 52 N/100 mm, at least 54 N/100 mm, at least 56 N/100 mm, at least 58 N/100 mm, at least 60 N/100 mm, at least 65 N/100 mm, or at least 70 N/100 mm.

16. A process for treating a surface comprising:
(a) providing a body;
(b) contacting the surface with a formulation to form a layer, the formulation comprising a phenol selected from the group consisting of 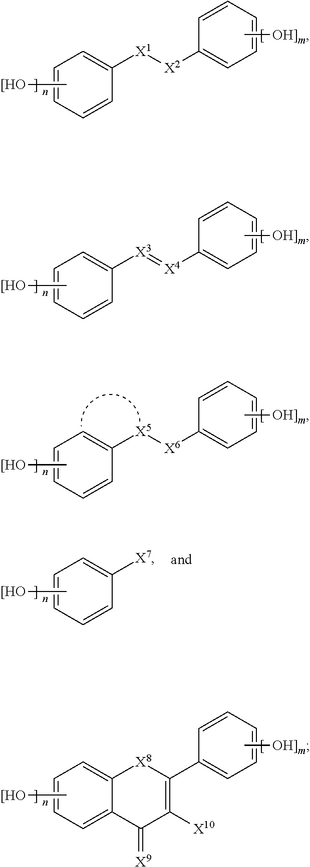
wherein n or m=0, 1, 2, 3, 4, 5, and n+m≥2;
X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer;
X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer;
X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer;
X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer;
X⁵ is a three, four, or five membered linker forming a fused bicyclic moiety in the phenol;
X⁶ is a single bond, a methylene, an ethylene, a propylene, —O(CH₂)ₜ—, —CH₂O(CH₂)ₜ—, —OCH₂(CHR⁵)(CH₂)ₜ—, —CH₂O(CHR⁵)(CH₂)ₜ—, —OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer;
X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, an amino alkylene group, a hydroxyl group, a hydroxyl alkylene group, an alkoxy group, an alkoxy alkylene group, a thiol group, a thiol alkylene group, an alkylthio group, or an alkylthio alkylene group;
X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl;
(c) curing the layer to form a coating onto the surface,
wherein the coating comprises a property selected from the group consisting of
(i) the coating has a water contact angle θ_{C} of equal or greater than 0° and not greater than 65° as determined by ASTM D7334-08, and
(ii) the coating has a tape test rating as determined by ASTM D3359-17 of at least 2B.

17. A process for treating a surface comprising:
(a) providing a first body;
(b) contacting the surface with a formulation to form a layer, the formulation comprising a phenol selected from the group consisting of 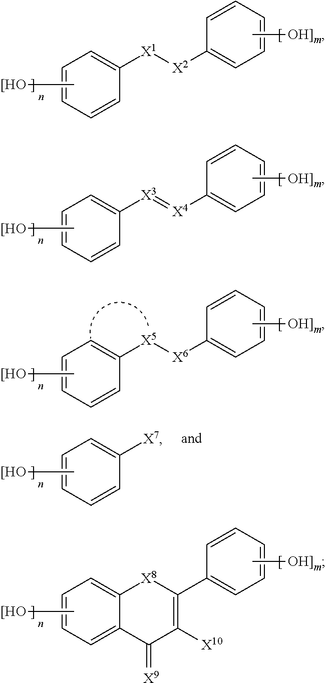
wherein n or m=0, 1, 2, 3, 4, 5, and n+m≥2;
X¹ is selected from —CHR¹(CH₂)ₚ—, wherein R¹ is hydrogen, a carboxyl group, a sulfonate group, a phosphonate group, an amino group, a hydroxyl group, a thiol group, or a halogen, p is 0 or an integer;
X² is a single bond, —OC(O)(CH₂)_{q}—, —C(O)O(CH₂)_{q}—, —OC(O)(CHR²)(CH₂)_{q}—, —C(O)O(CHR²)(CH₂)_{q}—, —OC(O)CR²═CR²—, wherein R² is independently for each occasion hydrogen, a carboxyl group, a sulfonate group, a phosphonate group, an amino group, a hydroxyl group, a thiol group, or a halogen, q is 0 or an integer;
X³ is selected from —(CH₂)ᵣCR³═, wherein R³ is hydrogen, a carboxyl group, a sulfonate group, a phosphonate group, an amino group, an hydroxyl group, a thiol group, or a halogen, r is 0 or an integer;
X⁴ is ═CR⁴OC(O)(CH₂)ₛ—, ═CR⁴C(O)O(CH₂)ₛ—, ═CR⁴OC(O)(CHR⁴)(CH₂)ₛ—, ═CR⁴C(O)O(CHR⁴)(CH₂)ₛ—, ═CR⁴(CH₂)ₛ, wherein R⁴ is independently for each occasion hydrogen, a carboxyl group, a sulfonate group, a phosphonate group, an amino group, a hydroxyl group, a thiol group, or a halogen, s is 0 or an integer;
X⁵ is a three, four, or five membered linker forming a fused bicyclic moiety in the phenol;
X⁶ is a single bond, a methylene, an ethylene, a propylene, —O(CH₂)ₜ—, —CH₂O(CH₂)ₜ—, —OCH₂(CHR⁵)(CH₂)ₜ—, —CH₂O(CHR⁵)(CH₂)ₜ—, —OC(O)(CH₂)ₜ—, —C(O)O(CH₂)ₜ—, —OC(O)(CHR⁵)(CH₂)ₜ—, —C(O)O(CHR⁵)(CH₂)ₜ—, wherein R⁵ is hydrogen, a carboxyl group, a sulfonate group, a phosphonate group, an amino group, a hydroxyl group, a thiol group, or a halogen, t is 0 or an integer;
X⁷ is selected from a halogen, a sulfonate group, a carboxylate group, a phosphonate group, an amino group, a nitro group, a —CH═CHR⁶ group, a —C(O)R⁶ group, a —CH(OH)R⁶ group, wherein R⁶ is selected from hydrogen, a C₁-C₆ alkyl group, a carboxyl group, a sulfonate group, a phosphonate group, an amino group, an amino alkylene group, a hydroxyl group, a hydroxyl alkylene group, an alkoxy group, an alkoxy alkylene group, a thiol group, a thiol alkylene group, an alkylthio group, or an alkylthio alkylene group;
X⁸ and X⁹ are independently selected from O, S, NH, or NR⁷, wherein R⁷ is a C₁-C₆ alkyl; X¹⁰ is selected from H, OH, OR⁸, SH, SR⁸, or NH₂, NHR⁸, wherein R⁸ is a C₁-C₆ alkyl;
(c) contacting a second body with the layer;
(d) curing the layer to form an adhesion between the first body and the second body, wherein after curing the layer has one of the following properties
(i) the adhesion between the first body and the second body 90 degree peel resistance as determined according to ASTM D6862-11 of at least 38 N/100 mm,
(ii) the adhesion between the first body and the second body has a lap shear resistance as determined by ASTM D1002, D3163, D3165, or D5868 of at least 0.1 N/mm²,
(iii) the adhesion between the first body and the second body has a T-peel resistance as determined by ASTM D1876 of at least 60 N/100 mm, or
(iv) the adhesion between the first body and the second body has a 180 degree peel resistance as determined by ASTM D3330 of at least 38 N/100 mm.

18. The process according to any one claims of claims 16 and 17, wherein the formulation further comprises a salt selected from an alkaline metal halide, earth alkaline halide, or any combination thereof.

19. The process according to claim 18, wherein the salt is selected from the group consisting of LiF, LiCl, LiBr, LiI, NaF, NaCl, NaBr, NaI, KF, KCl, KBr, KI, MgF₂, MgCl₂, MgBr₂, MgI₂, CaF₂, CaCl₂, CaBr₂, CaI₂, BaF₂, BaCl₂, BaBr₂, BaI₂, and any combination thereof.

20. The process according to any one of claims 16 through 19, wherein the formulation further comprises a diluent.

21. The process according to claim 20, wherein the diluent is selected from the group consisting of water, an alkane, an aromatic, an alcohol, an ether, a ketone, an ester, and any combination thereof.

22. The process according to any one of claims 16 through 21, wherein X⁵ is selected from the group consisting of CH—CH₂—CH₂—CH₂—, N—CH₂—CH₂—CH₂—, CH—NH—CH₂—CH₂—, CH—CH₂—NH—CH₂—, CH—CH₂—CH₂—NH—, C═CH₂—CH₂—CH₂—, CH—CH═CH—CH₂, CH—CH₂—CH═CH—, C═CH—CH═CH—, N—CH₂—CH═CH—, N—CH═CH—CH₂—, C═N—CH₂—CH₂—, CH—N═CH₂—CH₂—, CH—NH—CH═CH—, C═CH—NH—CH₂—, CH—CH═N—CH₂—, CH—CH₂—N═CH—,
CH—CH═CH—NH—, CH—CH₂—CH═N—, C═N—CH═CH—, C═CH—N═CH₂—, CH═CH—CH═N—, CH—O—CH₂—CH₂—, CH—CH₂—O—CH₂—, CH—CH₂—CH₂—O—, CH—O—CH═CH—, C═CH—O—CH₂—, CH—CH═CH—O—, CH—S—CH₂—CH₂—, CH—CH₂—S—CH₂—, CH—CH₂—CH₂—S—, CH—S—CH═CH—, C═CH—S—CH₂—, CH—CH═CH—S—, N—CH₂—CH₂—, CH—NH—CH₂—, CH—CH₂—NH—, C═N—CH₂—, CH—N═CH—
CH—CH═N—, C═CH—NH—, CH—O—CH₂—, CH—CH₂—O—, C═CH—O—, CH—S—CH₂—, CH—CH₂—S—, C═CH—S—.

23. The process according to any one of claims 16 through 22, wherein the phenol is selected from the group consisting of piceatannol, rosmarinic acid, rosmarinic salt, a norlaudanosoline, 4-aminocatechol, dihydroxybenzaldehyde, dihydroxymandelic acid, 4-nitrocatechol, 4-chlorocatechol, 3,4-dihydroxyphenylpyruvate, epinephrine, norepinephrine, and any combination thereof.

24. A formulation comprising:
a phenol selected from the reaction product consisting of (i) a phenolic reactant and (ii) a connecting reactant, wherein the phenolic reactant is selected from” 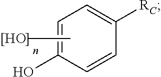
wherein n=0, 1, 2, or 3;
R_{C} is selected from hydrogen, (CH₂)ᵤC(O)R⁷ or (CH₂)ᵥR⁸, wherein R⁷ is selected from the group consisting of H, C₁-C₄ alkyl, OH, SH, Cl, Br, C₁-C₄-alkoxyl, or C₁-C₄-alkylmercaptyl, wherein R⁸ is selected from OH, SH, Cl, Br, C₁-C₄-alkoxyl, or C₁-C₄-alkylmercaptyl, wherein u and v are 0 or an integer;
wherein the reaction product includes at least two phenolic moieties;
wherein the phenol further comprises a property selected from the group of
(i) the phenol forms a coating when the formulation is applied to a surface and the coating has a water contact angle θ_{C} of equal or greater than 0° and not greater than 65° as determined by ASTM D7334-08,
(ii) the phenol forms an adhesion between a surface and an object having a 90 degree peel resistance as determined according to ASTM D6862-11 of at least 38 N/100 mm,
(iii) the phenol forms a coating when the formulation is applied to a surface and the coating has a tape test rating as determined by ASTM D3359-17 of at least 2B.
(iv) the phenol forms an adhesion between a surface and an object having a lap shear resistance as determined by ASTM D1002, D3163, D3165, or D5868 of at least 0.1 N/mm²,
(v) the phenol forms an adhesion between a surface and an object having a T-peel resistance as determined by ASTM D1876 of at least 60 N/100 mm,
(vi) the phenol forms an adhesion between a surface and an object having a 180 degree peel resistance as determined by ASTM D3330 of at least 38 N/100 mm.

25. The formulation according to claim 24, wherein the phenolic reactant is

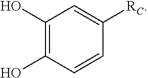

26. The formulation according to claim 24, wherein the connecting reactant is selected from an aldehyde, a ketone, a polyol, an amine, a polyamine, a hydroxyalkylamine, or a combination thereof and wherein the connecting reactant has a molecular mass of less than 400 g/mol, less than 380 g/mol, less than 360 g/mol, less than 340 g/mol, less than 320 g/mol, less than 300 g/mol, less than 280 g/mol, less than 260 g/mol, less than 250 g/mol, less than 240 g/mol, less than 230 g/mol, less than 220 g/mol, less than 210 g/mol, less than 200 g/mol, less than 190 g/mol, less than 180 g/mol, less than 170 g/mol, less than 160 g/mol, or less than 150 g/mol.

27. The formulation according to claim 24, wherein the connecting reactant is selected from the group consisting of: 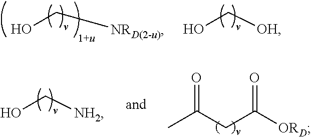
wherein v is an integer between 1 and 10; u is 1, or 2; and R_{D} is hydrogen or a C₁-C₄ alkyl group.

28. The formulation according to claim 24, wherein the phenol is selected from the group consisting of: 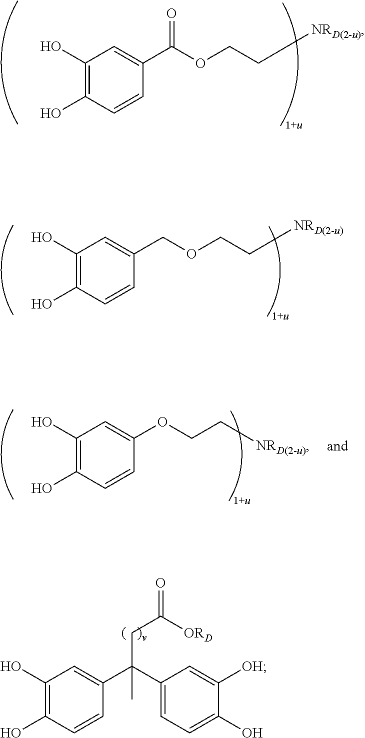
wherein v is an integer between 1 and 10; u is 1, or 2; and R_{D} is hydrogen or a C₁-C₄ alkyl group.
